(19) United States
(12) Patent Application Publication
(10) Pub. No.: US 2006/0233861 A1
(43) Pub. Date: Oct. 19, 2006

(54) Modulation of atp production or content in the hypothalamus

(76) Inventor: David B. Maclean, Middletown, RO (US)

Correspondence Address: MORGAN LEWIS & BOCKIUS LLP, 1111 PENNSYLVANIA AVENUE NW, WASHINGTON, DC, 20004, US

(21) Appl. No.: 10/545,677

(22) PCT Filed: Feb. 16, 2004
(86) PCT No.: PCT/GB04/00598

§ 371 Date: Feb. 28, 2006

Related U.S. Application Data
(60) Provisional application No. 60/447,454, filed on Feb. 14, 2003.

(51) Int. Cl. A61K 36/185; A61K 47/00; A61K 31/704

(52) U.S. Cl. 424/439; 424/725; 514/26

(57) ABSTRACT

A method for the identification of agents for use in the treatment of metabolic diseases, disorders or conditions, for example obesity and diabetes, or for causing weight loss without substantial adverse health effects, in an animal in need of such treatment, comprises the step of identifying an agent that modulates ATP production or content in the hypothalamus of an animal.

### FIELD OF THE INVENTION

The present invention relates to methods for treating metabolic diseases, disorders or conditions, for example appetitive diseases, disorders or conditions (e.g. obesity) and/or diabetes, or for causing weight loss without substantial adverse health effects, by modulating adenosine triphosphate (“ATP”) production or content in the hypothalamus.

### BACKGROUND

Medicinal plants reported in the folkloric or traditional medicine literature have long been a source for identifying new mechanisms for altering physiological or pathological responses. U.S. Pat. Nos. 6,376,657 and 6,488,967, the disclosures of which are incorporated herein by reference, disclose steroidal glycosides with anorectant activity in animals, for example, the steroidal glycisde depicted below as a compound of Formula “(1)”, which was isolated from the South African Hoodia plant and shown to have C-12 and C-14 homologies to the steroidal core of cardiac glycosides. 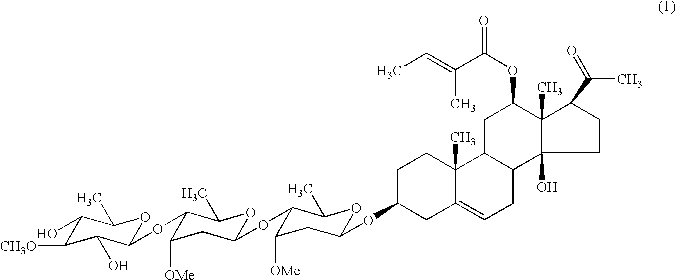
Although the core is similar in structure to the cardiac glycosides, initial attempts to identify binding or inhibition of activation of the compound of Formula (1) to known receptors or proteins, including the Na/K ATPase (the putative target of cardiac glycosides) were unsuccessful.

### BRIEF DESCRIPTION OF THE INVENTION

Applicants have discovered that the compound of Formula (1) antagonizes the ouabain-induced inhibition of cellular uptake of 86-Rubidium (an assay for Na/K ATPase activity) which was shown later to be an indirect effect mediated by increased availability of ATP, the primary energy substrate for maintaining Na/K ATPase pump activity.

Based on these findings, the present invention provides a method for the identification of agents for use in the treatment of metabolic diseases, disorders or conditions, for example appetitive diseases, disorders or conditions (e.g., obesity), or diabetes (Type 1 or 2), in an animal in need of such treatment, comprising the step of identifying an agent that modulates ATP production or content in the hypothalamus of an animal (the expression “modulates ATP production or content in the hypothalamus of the animal” including, but not limited to, increasing ATP production or content in the hypothalamus).

In a preferred embodiment, the present invention provides a method for the identification of agents that regulate food-intake or calorific-intake in an animal, comprising the step of identifying an agent that modulates ATP production or content in the hypothalamus of an animal.

In another preferred embodiment, the present invention provides a method for the identification of agents that decrease food-intake or calorific-intake in an animal, comprising the step of identifying an agent that increases ATP production or content in the hypothalamus of an animal.

In another preferred embodiment, the present invention provides a method for the identification of agents that cause body mass reduction in an animal without substantial adverse health effects, comprising the step of identifying an agent that modulates ATP production or content in the hypothalamus of the animal, including, but not limited to, increasing ATP production or content in the hypothalamus of the animal.

In another embodiment of the present invention, a method is provided for the identification of agents for use in the treatment of diabetes in an animal in need of such treatment, comprising the step of identifying an agent that modulates ATP production or content in the hypothalamus of the animal including, but not limited to, increasing ATP production or content in the hypothalamus of the animal.

In accordance with a further aspect of the present invention, after identification of an agent that modulates ATP production or content in the hypothalamus of an animal, a source of sufficient quantities of the agent is then identified. Such a source may suitably be a plant or a plant extract, or alternatively a synthetic route to the agent may be identified. The synthetic route may, if appropriate, use natural (e.g. plant-derived) starting materials. The plant may, if desired, be processed before use, e.g. dried or otherwise preserved, comminuted and/or mixed with other components to form a composition. The plant extract may, if desired, be purified and/or isolated from the remainder of the plant material before use, and/or may be mixed with other components to form a composition.

The plant extract may be obtained by any appropriate method for extracting a pharmacologically active agent from a plant. The method may include, for example, crushing, drying (e.g. freeze-drying, spray-drying or vacuum-drying), cutting, mashing, cold water extraction, hot water extraction, organic solvent extraction, fractionation, chromatography, distillation, filtration, centrifugation, liquefied gas extraction such as carbon dioxide extraction, pressing, stirring and/or washing.

The agent or source thereof may be used as such or may be formulated into a composition prior to administration to the animal. When present in a composition, the agent will typically be in association with one or more excipient, diluent or carrier. Any convenient composition form may be used, including pharmaceutical (including veterinary) compositions, foodstuffs, food additives, beverages and beverage additives.

The plants used are preferably ethically produced, and in particular are preferably farmed under controlled conditions to preserve wild popluations. The controlled farming conditions preferably include treatment of fungal or other infections to enhance the quality and growth rate of the plants and to maximise the yield of the pharmacologically active agents.

In another embodiment of the present invention, a method for treating a metabolic disease, disorder or condition, e.g. an appetitive disease, disorder or condition (e.g., obesity), in an animal in need of such treatment is provided, comprising the step of administering a therapeutically effective amount of an agent known through (e.g. public) scientific research to modulate ATP production or content in the hypothalamus of the animal, and for the purpose of knowingly modulating ATP production in the hypothalamus of the animal, including, but not limited to, increasing ATP production or content in the hypothalamus of the animal.

Accordingly, the present invention also provides the use of a therapeutically effective amount of an agent known through (e.g. public) scientific research to modulate ATP production or content in the hypothalamus of an animal for the preparation of a medicament for treating a metabolic disease, disorder or condition, e.g. an appetitive disease, disorder or condition (e.g., obesity) in the animal in need of such treatment by knowingly modulating ATP production or content in the hypothalamus of the animal, including, but not limited to, increasing ATP production or content in the hypothalamus of the animal.

In another embodiment of the present invention, a method for treating obesity in an animal in need of such treatment is provided, comprising the step of administering a therapeutically effective amount of an agent known through (e.g. public) scientific research to modulate ATP production or content in the hypothalamus of the animal, and for the purpose of knowingly modulating ATP production or content in the hypothalamus of the animal, including, but not limited to, increasing ATP production or content in the hypothalamus of the animal.

Accordingly, the present invention also provides the use of a therapeutically effective amount of an agent known through (e.g. public) scientific research to modulate ATP production or content in the hypothalamus of an animal for the preparation of a medicament for treating obesity in the animal in need of such treatment by knowingly modulating ATP production or content in the hypothalamus of the animal, including, but not limited to, increasing ATP production or content in the hypothalamus of the animal.

In yet another embodiment of the present invention, a method for treating diabetes in an animal in need of such treatment is provided, comprising the step of administering a therapeutically effective amount of an agent known through (e.g. public) scientific research to modulate ATP production or content in the hypothalamus of the animal, and for the purpose of knowingly modulating ATP production or content in the hypothalamus of the animal, including, but not limited to, increasing ATP production or content in the hypothalamus of the animal.

Accordingly, the present invention also provides the use of a therapeutically effective amount of an agent known through (e.g. public) scientific research to modulate ATP production or content in the hypothalamus of an animal for the preparation of a medicament for treating diabetes in the animal in need of such treatment by knowingly modulating ATP production or content in the hypothalamus of the animal, including, but not limited to, increasing ATP production or content in the hypothalamus of the animal.

In yet another embodiment of the present invention, a method for causing body mass reduction (weight loss) in an animal without substantial adverse health effects is provided, comprising the step of administering a therapeutically effective amount of an agent known through (e.g. public) scientific research to modulate ATP production or content in the hypothalamus of the animal, and for the purpose of knowingly modulating ATP production or content in the hypothalamus of the animal, including, but not limited to, increasing ATP production or content in the hypothalamus of the animal.

Accordingly, the present invention also provides the use of an therapeutically effective amount of an agent known through (e.g. public) scientific research to modulate ATP production or content in the hypothalamus of an animal for the preparation of a medicament for causing body mass reduction (weight loss) in the animal without substantial adverse health effects by knowingly modulating ATP production or content in the hypothalamus of the animal, including, but not limited to, increasing ATP production or content in the hypothalamus of the animal.

In each of these embodiments, it is the premise that the hypothalamus ‘senses’ ATP content as a means to monitor whole animal energy status. Food intake is regulated accordingly—i.e. when ATP production/content is increased, the hypothalamus senses that body energy stores are adequate. ATP is the primary source of energy for almost all energy requiring chemical reactions in the body. The embodiment also assumes that ATP is a major mediator of subsequent signaling by the hypothalamus, e.g. by activating ATP-dependent ion channels or changing the activity of the Na/K ATPase.

The present invention does not embrace activities taught in the prior published art or obvious therefrom. In particular, excluded from the scope of the present invention are the acts of suppressing appetite, treating metabolic disorders, diseases or conditions, causing body mass reduction and treating diabetes, and acts associated with preparing medicaments and other compositions therefor, other than in the context of known and intended modulation of ATP production or content in the hypothalamus, including, but not limited to, increasing ATP production or content in the hypothalamus. In particular, the use of agents which are extracts from plants of the genus Trichocaulon or the genus Hoodia is not within the present invention unless it is in the context of known, expected and intended modulation of ATP production or content in the hypothalamus.

### Definitions

The phrase “therapeutically effective amount” means an amount of an agent that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), laboratory animals, food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventive, i.e., prophylactic, and palliative treatment.

As used herein, the term “ATP modulator” refers to an agent that modulates (up, down or neutrally, including—but not limited to—in a regulatory manner) the production of ATP in the hypothalamus of an animal. An agent is any chemical substance that produces the desired effect. The described increase in the content or production of ATP in the hypothalamus is therefore an example of ATP modulation.

The abbreviations “ATP” and “ATPase” through out the specification refer to their accepted meanings in the art—ATP represents adenosine triphosphate; and ATPase represents adenosine triphosphatase.

### DESCRIPTION OF THE FIGURES

FIG. 1A and FIG. 1B illustrate the effect of ICV administration of test compounds on rat food intake. The test compounds or dimethylsulfoxide (DMSO) vehicle were administered by microsyringe in 4 μl volumes and food intake measured for the subsequent 24 hour period. N=6 animals per group, * P<0.05 vs vehicle control. Compound (1) is represented in FIG. 1A and Compound (2) is represented in FIG. 1B.

FIG. 2A, FIG. 2B and FIG. 2C illustrate 86-Rubidium uptake in cultures of fetal rat hypothalamic neurons. Two congeners of the Hoodia plant extracts (Compounds (1) and (2)) were tested for their effect on ouabain inhibition of presumptive Na/K ATPase function as determined by ⁸⁶Rb uptake. N=8 wells per group (pooled experiments), * P<0.05. FIG. 2A illustrates the effect of ouabain alone; FIG. 2B illustrates the effect of ouabain alone, Compound (1) alone, or the two agents combined on rubidium uptake; FIG. 2C illustrates ouabain plus Compound (2).

FIG. 3 illustrates the effect of Compound (1) on the ouabain effect on 86-rubidium uptake into rat hypothalamic slice punches. Punches were obtained and incubated as described in the text. N=6 samples per group, * P<0.05

FIG. 4 illustrates the effect of Compound (1) on ^{3H}ouabain binding in cultured fetal rat hypothalamic neurons. Cultures were maintained then exposed to ^{3H}ouabain, ouabain and/or Compound (1) as described in the text. N=6 wells per group, * P<0.05.

FIG. 5 illustrates the effect of Compound (1) on the ATP content in cultured fetal hypothalamic neurons. Cultures were exposed to ouabain, Compound (1) or the two combined as described in the text. N=5-6 samples per group, * P<0.05 vs control or ouabain alone

FIG. 6 illustrates the effect of ICV Compound (1) (40 nmoles) or DMSO vehicle on ATP content in hypothalamic slice punches removed from ad lib diet fed rats. Rats were treated ICV and slice punches obtained 24 hours later. N=6 rats per group, * P<0.05

FIG. 7 illustrates the effect of a 4 day hypocaloric (5 gm/day) diet in rats on ATP content in brain regions and liver. N=6 rats per group, * P<0.05 vs control fed animals.

FIG. 8 illustrates the effect of ICV vehicle or Compound (1) (40 nmol) on ATP content in hypothalamic slice punches removed from rats maintained on a hypocaloric diet. N=6 rats per group, * P<0.05 vs vehicle-treated or control-fed rats.

### DETAILED DESCRIPTION OF THE INVENTION

There has been a resurgence of interest in traditional and folkloric medicines as possible therapeutic agents as well as clues or leads to novel therapeutic mechanisms. An example of such an anecdotal or folkloric agent is the sap from a group of South African plants of the species Hoodia (including subspecies H. Gordonii or H. Lugardii), a member of the large milkweed family. Based on a limited number of reports from and subsequent interviews with both native and foreign South Africans, the sap apparently assuaged both the feelings and sensations (e.g. ‘pangs’) of hunger. Bruyns, P., “A Revision of Hoodia and Lavrania (Ascelepidaceae-Stapeliaeae, Botanische Jahrbucher:furer)”, Systematik Pflanzengeschichte und Pflanzengeographie, 115, 145-270 (1993). Several active components have been isolated and identified, in particular, those described in U.S. Pat. Nos. 6,488,967 B1 and 6,376,657 B1, both of which are incorporated herein by reference. Dried extracts of the plant sap, which contain not only the putative active agent but multiple other components, have been administered long term to several species, including the diabetic obese Zucker rat. In those experiments, anorectant activity and reversal of diabetes are maintained for the duration of dosing, up to 8 weeks; other studies demonstrate that the food inhibition and weight loss are independent of diet type. In addition, short-term studies in humans have recently been disclosed which support these findings.

The putative active component in these sap extracts is a tri-rhabinoside, 14-0H, 12-tigloyl pregnane steroidal glycoside (referred to herein as “Compound (1)” and having Formula (1)). 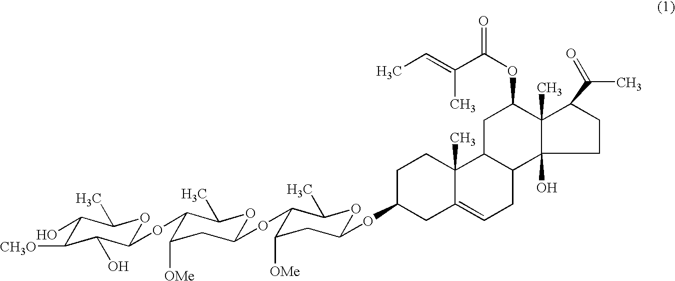
The core steroid, particularly regarding the 14-OH substitution, is somewhat similar to other cardenolides (1-3). See, e.g., Tschesche, R., (1972) Proc R Soc Lond B Biol Sci, 180, 187-202; Gupta, S. P., (2000) Prog Drug Res, 55, 235-282; and Kren, V., et al., (2001) Curr Med Chem, 8, 1303-1328. The isolation and purification of Compound (1) is described in U.S. Pat. No. 6,376,657 B1, incorporated herein by reference.

The studies described herein led to the key finding that Compound (1) increases the production (and/or content) of ATP within hypothalamic neurons, either in whole cell cultures of fetal neurons or in hypothalamic slice punches from adult animals. Furthermore, Applicant found a reduction in hypothalamic ATP in rats after 4 days of moderate (75%) food deprivation (5 g/day)—and the ‘reversal’ of those ATP reductions in rats treated with intracerebroventricular (VIII) injections of Compound (1). Based on these findings, it is reasonable to believe that a fundamental mechanism of hypothalamic regulation of food intake is altered intracellular concentrations of ATP. Thus, the sensing of energy homeostasis, as signaled by intracellular ATP, may be directly analogous to other fundamentally simple but tightly regulated hypothalamic homeostatic mechanisms, in particular those controlling osmolarity and body temperature. See, e.g., Reichlin, S. “Function of the Hypothalamus”, Am. J. Med., 43, 477-485 (1967); Schrier, R. W., et al., (1979) Am J Physiol, 236, F321-332; Loewy, A., et al., (1980) Federation Proceedings, 39, 2495-2503; and Benzinger, T. (1969) Physiological Reviews, 49, 671-759. This finding also supports a large body of evidence that the hypothalamus is the principle integrator and controller of the organism's overall energy balance. See, e.g., Mayer, J., et al., “Regulation of Food Intake and Obesity,” Science, 156, 328-337 (1967); Bray, G. A. (1980) Int J Obes, 4, 287-295; and Levin, B. E., et al., (1996) Am J Physiol, 271, R491-500.

Both dried whole sap and the purified plant extracts of the plant Hoodia Gordonii were known from previous work to be active using both per os, subcutaneous, and intravenous routes of administration. Using relatively small amounts of material (0.4 to 40 nmoles) injected ICV suggested but did not prove that at least one locus of action is within the CNS. However, intraperitoneal injections of amounts that were very active when injected ICV had no discemable effect on food intake. That the compound is active centrally, however, by no means excludes that it may also have peripheral effects on appetite regulation, for example by effects on vagal afferent nerves (see, MacLean, D. (1985) Regul Pept, 11, 321-333) direct effects on gastric emptying (versus those mediated by CNS effects on vagal efferent function), or effects on potentially anorectant peripheral hormones, such as CCK.

The core structure of these Hoodia-derived compounds has similarities to the cardiac glycosides or cardenolides, both the general planar structure of the steroidal core, the 14-OH group and the glycoside side chain. In addition, the Hoodia compounds have a relatively bulky C-12 substitution, normally hydroxylated on cardenolides. Thus the initial hypothesis was that these compounds may in some way interact with the Na/K ATPase within the brain, and that such effects might in turn alter hypothalamic function by either direct effects on dopamine metabolism (see, Taglialatela, M., et al., (1988) Journal of Pharmacology & Experimental Therapeutics, 246, 682-688) glucose sensitive neuron thresholds (see, Angel, I., et al., (1985) Proc Natl Acad Sci U S A, 82, 6320-6324; Levin, B. E. (2001) Int J Obes Relat Metab Disord, 25 Suppl 5, S68-7221; and Levin, B. E., et al., (2001) Nat Neurosci, 4, 459-460) or altered consumption of intracellular ATP (see, Tsuchiya, K., et al., (1998) J Cardiovasc Electrophysiol, 9, 415-422). In addition, there are persistent reports of endogenous ouabain-like cardiac glycosides within the hypothalamus (see, Sancho, J. M. (1998) Clin Exp Hypertens, 20, 535-542; and Schoner, W. (2000) Exp Clin Endocrinol Diabetes, 108, 449-454) for which Compound (1) might conceivably act as an exogenous antagonist.

Most of the in vitro data, however, does not support those hypotheses. In multiple Na/K ATPase assays with membrane extracts of brain, gut, and kidney, no evidence was obtained supporting either direct effects or inhibition of ouabain effects on directly measured Na/K ATPase activity. In addition, other studies demonstrated no evidence of interference with tritiated ouabain binding in whole cell preparations of neuronal cultures. Rather, it was demonstrated below in two whole cell systems—hypothalamic neuronal cultures and incubated hypothalamic punch explants from adult rats-a likely functional antagonism of the ouabain inhibition of 86-rubidium uptake. That assay has been a well-characterized system to measure Na/K ATPase activity, inhibition of which leads to gradual depletion of intracellular potassium (manifested by reduced 86-rubidium uptake) and accumulation of sodium.

However, there are other possible explanations for the apparent preservation of intracellular potassium in the face of inhibitory concentrations of ouabain. The most important of these is the closure of K+ channels, blocking the efflux of potassium and thus resulting in the accumulation of 86-rubidium (see, Tsuchiya, K., et al., (1998) J Cardiovasc Electrophysiol, 9, 415-422; and Miki, T., et al., (2001) Nat Neurosci, 4, 507-512). There is evidence of electrophysiological data that Compound (1) does indeed activate (close) ATP-sensitive K+ channels (see, Ashcroft, F. M. (1988) Annu Rev Neurosci, 11, 97-118; and Ashcroft, S. J., et al., (1990) Cell Signal, 2, 197-214) in glucose sensitive neurons—based on recordings in hypothalamic slice recordings, an effect we believe is mediated by increased ATP (see below). Experiments have not yet been performed to rule out this activity as the cause of the apparent ouabain antagonism. In the experimental conditions described below, test compounds were added first, then vehicle or ouabain, and finally 86-rubidium. Thus, it is somewhat unlikely that continued Compound (1) closure of ATP-sensitive K+ channels would offset the effect of ouabain on promoting passive K+ efflux.

Instead, it is believed that apparent effects on both the ATP-sensitive K+ channel and the inhibition of ouabain activity in whole cell but not membrane preparations might be secondary to a common secondary mechanism, specifically increased intracellular ATP—ATP being the principle rate-limiting regulator of pump activity. This hypothesis was tested by direct measurement of ATP content in tissue culture, explants of slice punches and slice punches following in vivo treatment with Compound (1). See, Example section below. In each of those conditions there were consistent increases in total ATP content. There is one report of changes in directly measured hypothalamic content of ATP (see, Gray, J. M., et al., (1998) Brain Res, 798, 223-231) in gonadal steroid treated rats, but no studies described in altered nutritional status. To attempt to physiologically manipulate ATP content, an experiment previously reported by Ji, H. et al. in Physiol Behav, 68, 181-186 (1999) was repeated in which reduced hepatic ATP content was induced with a hypocaloric diet. In addition to liver, ATP content was measured in several brain regions and in dissected hypothalamus. The experiments confirmed the reduced hepatic ATP content following hypocaloric diet, reported by Ji et al. (ibid). In addition, reduced content in dissected hypothalamus was found, but not in cerebellum or cortex. In animals pair fed for 4 days and treated a day prior to sacrifice, ATP content was increased in the hypothalami from Compound (1) versus vehicle treated animals. Similar evidence of hypothalamic ATP apparent depletion' was also observed in another model of relative caloric deficiency, rats treated with supraphysiological doses of triiodothyronine (T3) (see, Luo, L., et al., (2002) 84th Ann Mtg Endocrine Soc Abstracts, OR53-6, p 136). In that model, the apparent ATP deficit was also partially reversed by ICV Compound (1).

Until recently, there were few studies attempting to measure ATP or ATP/ADP ratios in whole brain. MR spectroscopy has been increasingly used for this purpose, to date in predominantly pathological states such as brain trauma, mental illness or recurrent seizures (see, Ross, S. T., et al., (2000) J Neurophysiol, 83, 2916-2930; and Tanaka, K., et al., (1997) Science, 276, 1699-1702).

At the present time, there does not appear to be a mechanistic explanation for the observed changes in ATP following Compound (1) treatment. Based on the in vitro results, the mechanism is likely to be ‘local’ or intracellular rather than due to a whole organism integrative or hormonal response (see, Kalra, S. P., et al., (1999) Endocr Rev, 20, 68-100). A specific molecular target or pathway has not been identified, such as an indirect effect on intracellular substrate oxidation or conversely decreased ATP consumption. However, based on the effects of cardiac glycosides on Na/K ATPase, it is reasonable to believe that Compound (1) may have a direct effect on another ATPase regulating ATP balance. For example, there is limited evidence that other steroids, e.g. estrogen, may directly regulate the F1/F0 ATP synthase (see, Vinogradov, A. D. (2000) J Exp Biol, 203 Pt 1, 41-49; Zheng, J., et al., (1999) J Steroid Biochem Mol Biol, 68, 65-75; and Zheng, J., et al., (1999) Eur J Pharmacol, 368, 95-102). Natural product and semisynthetic inhibitors of the mitochondrial ATP synthase are well characterized (see, Salomon, A. R., et al., (2000) Proc Natl Acad Sci U S A, 97,14766-14771).

A recent paper by Ji et al. (ibid) suggested that the rate and subsequent termination of re-feeding following prolonged fasting in rats is directly correlated with feeding-induced hepatic replenishment of ATP. The findings described below complement that study and support the concept that the hypothalamus may also be a sensing target organ that is vulnerable to ATP fluctuations. Two recent reports also support the concept that metabolic activity of hypothalamic neurons directly regulates food intake (see, Xin, X., et al., (2000) Brain Res Bull, 52, 235-242; and. Wang, H., et al., (1999) Brain Res, 843, 184-192). Loftus and co-workers in Science, 288, 2379-2381 (2000) demonstrated that ICV administration of a fatty acid synthase (FAS) inhibitor, C-75, reduced food intake and also inhibited NPY expression. The result suggested that FAS inhibition mimicked a caloric replete state. Similar results occur with oleic acid infusions into the hypothalamus (see, Obici, S., et al., (2002) Diabetes, 51, 271-275)

The hypothalamus is also sensitive to insulin and glucose, the latter transducing its effects through the ATP-sensitive K+ channel (Levin, B. E., et al., (2001 Nat Neurosci, 4, 459-460; Debons, A. F., et al., (1977) Fed Proc, 36, 143-147; and Levin, B. E., et al., (1999) Am J Physiol, 276, R1223-1231). These KATP channel functions are analogous, but not necessarily identical to glucose oxidation-dependent regulation of the pancreatic beta cell (see, Schuit, F. C., et al., (2001) Diabetes, 50, 1-11) or cardiac myocye (see, Knopp, A., et al., (2001) Cardiovasc Res, 52, 236-245). In a recently reported knockout of the gene encoding the ATP-sensitive K+ channel, the KO mice did not respond to hypoglycemia with increased food intake and were also heavier than wild type (see, Miki, T., et al, (2001) Nat Neurosci, 4, 507-512). Leptin signaling also may be partly mediated through the ATP-dependent K channel (see, Spanswick, D., et al., (1997) Nature, 390, 521-525); ATP content, as a regulator of that channel, may be one reason for the altered leptin sensitivity described in obesity (see, Koyama, K., et al., (1998) J Clin Invest, 102, 728-733; and Levin, B. E., et al., (2002) Am J Physiol Regul Integr Comp Physiol, 283, R941-948) and hypothyroid states (see, Iossa, S., et al., (2001) Int J Obes Relat Metab Disord, 25, 417-425).

ATP is also a putative neurotransmifter within the hypothalamus, for example, for studies regarding the neuroendocrine responses to stress (see, Wallace, R. A., et al., (2001) Clin Exp Rheumatol, 19, 583-586; Buller, K. M., et al., (1996) Neuroscience, 73, 637-642; and Kapoor, J. R., et al., (2000) J Neurosci, 20, 8868-8875) or studies regarding regulation of body temperature (see, Gourine, A. V., et al., (2002) Br J Pharmacol, 135, 2047-2055). While ATP may have direct effects on K+ channel activity or Na/K ATPase, many other phosphorylation-dependent transduction pathways may mediate the subsequent integrative response to energy sensing.

In summary, the present invention further extends the concept of hypothalamic nutrient and energy sensing and suggests that neurons within the medial basal hypothalamus may be vulnerable to oscillations of and thus sensitive to altered content of ATP. The steroidal glycosides (e.g., Compounds (1) and (2)) may also provide a useful probe to further elucidate the amplification of signaling of this energy sensing function.

### EXAMPLES

The test compounds (plant steroidal glycoside Compounds (1) and (2)) used in the following examples were prepared using the procedures described in U.S. Pat. No. 6,376,657.

The chemical structure for Compound (1) is described above. The chemical structure for Compound (2) is depicted below.

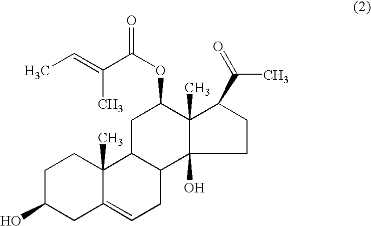

Rubidium-86 (⁸⁶Rb) was purchased from Amersham™ (United Kingdom). The Enliten™ ATP Assay kit was purchased from Promega™ (Madison, Wis.).

Male S.D. rats weighing an average 170±5 g were purchased from Charles River Laboratories (Wilmington, Mass.). The rats were housed individually in metabolic cages in a temperature-controlled environment (22° C.) with 12-h light/12-h dark cycle. All rats had ad libitum access to pelleted rat chow and tap water except when noted otherwise. Animals were weighed daily or as appropriate and daily food intake were measured using standard metabolic cage techniques. The Animal Care Committee at the Rhode Island Hospital approved the study protocols used in these experiments.

Intracerebroventricular (ICV) (III Ventricle) Injections:

In several experiments, to determine subsequent effects on food intake, hypothalamic Na/K ATPase activity or ATP content, the animals were administered the test compounds by ICV injection. Animals were anesthetized with sodium barbiturate (6 mg/100 g BW). Under stereotaxic fixation (David Kopf), with aseptic technique, a 25 ul Hamilton micro syringe (Reno, Nev.) needle tip was inserted according to the published references: 2 mm lateral to the middline at the bregma; −4.3 mm to the bregma; 5.5 mm deep to the dura; −1.3 mm with the shaft inclined medially at 13° C. to the vertical. The test Compound was dissolved in DMSO to form a 20 mM stock concentration, and 4 ul (or vehicle) was microinjected into the Third (III) Ventricle. All injections were calculated and expressed in absolute amount, i.e. nmoles. After rats recovered from anesthesia, they were returned to individual cages and food intake measurements continued for 24 hour intervals until the end of the experiment.

At the end of each experiment, the animals were administered a lethal anesthetic overdose prior to dissection and subsequent ex vivo assay of brain tissue.

In Vivo Experiments:

Food intake was controlled in the caloric deprivation study by pre-weighing fixed amounts of powdered chow (5.0 grams) and supplying it on a daily basis.

Hypothalamic Slice Punch, ⁸⁶Rb Uptake:

The whole brain was removed from the skull and inserted into a Precision Brain Slicer (RMB 3000C, Braintree Scientific INC.). As scaled from the rear, a brain slice was removed between the 8 and 10 scale (about 2 mm). The medial-basal hypothalamus was punched using a round mouth #18 1 mm diameter needle with attached syringe. The tissue included most of arcuate nucleus of the hypothalamus and a small part of nucleus paremamillaris ventralis. The tissue was pushed into 0.5 ml DMEM medium in a 24 well cell culture plate. Following preincubation at 37° C., in air/5% CO₂ for 30 min., concentrations of test Compound were added for an additional 30 min. Finally, 86-rubidium- (⁸⁶Rb) 1 μCi was added to each well for 20 minutes at room temperature. The tissue was then transferred to tubes with a protein dialysis solution to dissolve protein. Five (5) ml scintillation liquid was added to each tube and the photoemissions determined in a β-counter (Beckman).

To measure the effect of the test compound on hypothalamic ATP in diet-controlled hypocaloric rats, hypothalamic slice punches were prepared as described above 24 hours following the test compound ICV injection. Following extraction, the content of ATP in these slice punches was measured by the Enliten™ ATP Assay system (see below). In some studies, in order to parallel the 86-rubidium conditions, the punches were maintained in vitro as described above for a 20-minute incubation period at a final test concentration of 100-500 nM.

Hypothalamic Neuronal Culture:

Primary rat hypothalamic cells were prepared from fetal rats (Day 17) using methods described by Luo, L. G., et al., in Endocrinology, 136, 4945-4950 (1995). Briefly, the hypothalamus was removed and dispersed enzymatically for 2 hours with neutral protease in culture medium at a concentration of 100 U/dl. The dispersed cells were then plated at a low density of 10⁶ cells/ml in 24-well culture dishes pre-coated with 20 μg/ml poly-D-lysine (Sigma). The cells were maintained in bicarbonate buffered Dulbecco's modified Eagle's Medium (DMEM) supplemented with 10% fetal calf serum (FCS), at 37° C., 5% CO₂ and 95% humidity.

For the described experiments, hypothalamic neurons were pre-cultured for 11 days to allow maturation of a relatively pure neuron culture. Before testing the effect of the test compounds, neurons were washed once with a buffered salt solution 0.01 M Tris.HCl-0.15 M NaCl (pH 7.4). In initial determination of the IC50 for ⁸⁶Rb uptake, ouabain was added at concentrations of 10, 100, 1000, 10000, and 100,000 nM. For subsequent experiments, ouabain was added to a final concentration of 1000 nM (the approximate IC50). For testing the effect of the test compound, neurons were preincubated at selected concentrations in 0.4 ml DMEM for 10 minutes, and then ouabain was added to a final concentration of 1000 nM for an additional 20 minutes at 37° C. Finally, ⁸⁶Rb (Amersham) 1.0 μCi was then added to the wells, which were kept at room temperature for another 20 minutes. Medium was removed and neurons were triple washed with ice-cold NaCl solution to remove free ⁸⁶Rb. The cells were then lysed in 100 μl 0.05% Triton-100 in PBS and the lysate harvested with an additional 0.5 ml PBS. The lysate was added to 5 ml scintillation fluid and ⁸⁶Rb photon emissions counted for a minimum of 5 minutes.

Measurement of ATP Content:

The Enliten™ ATP Assay system with bioluminescence detection kit (Promega Madison, Wis.) was used for ATP measurement. The principle of the assay is that luciferase from Photinus pyralis catalyses D-luciferin in the presence of ATP and oxygen to oxyluciferin, Pi, AMP, carbon dioxide, and light.

As described above, hypothalamic tissues after removal or removal/incubation were transferred to 1.5 ml Eppendorf tubes with 50 μl TCA (5%) and immediately homogenated with a plastic homogenizer. The suspension was spun at 2000 rpm and the supernatant stored at −80° C. until assay. ATP was measured according to the kit protocol. Briefly, samples were neutralized to pH 7.4 with 10 μl 4 M Tris and 10 μl added to a new tube with 90 μl ATP-free water. The luciferase reagent was added 1 second before a 5 seconds measurement in the luminometer, as described by the supplier. Light photons were measured by a luminometer and were compared with an ATP standard curve to calculate ATP concentration. See, e.g., Levy, J. R., et al., (2000) Am J Physiol Endocrinol Metab, 278, E892-901). ATP content is expressed as moles per mg protein for tissue samples, moles per 5 mg tissue for section punches, and as moles per neuron culture well; expressed in appropriate quantitative units for each experiment.

Statistics:

In the text and figures, all data are presented as means ±SEM. Most described experiments have been repeated three times. However, for figures, the results of one experiment are shown. Food intake and body weight data used for graphical presentation and statistical analysis are expressed as per experimental time periods or for the 24 hours following ICV injection. Food intake, body weight, ATP content and ⁸⁶Rb uptake data were analyzed by ANOVA statistics program using a two factor analysis of variance of repeated measures. Post hoc comparisons among individual means were made by Tukey's t test.

### Results

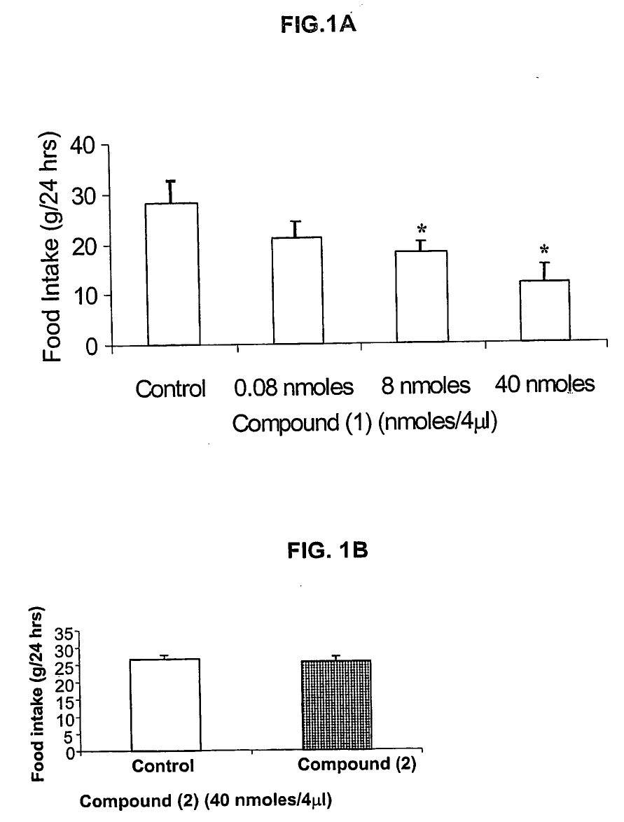

Effect of ICV P57s and Other Steroidal Glycosides on Food Intake During 24 Hour Periods (FIG. 1):

To determine whether the anorectant activity may occur within the brain, specifically within the hypothalamus, test compounds were injected into the third ventricle under stereotactic guidance at doses of 0.4 to 40.0 nmoles, dissolved in 4 μl DMSO. DMSO alone was injected as the control.

As shown in FIG. 1A for one representative study, in multiple experiments, Compound (1) reduced food intake over the 24 hours following ICV injection by 50-60%. The effect was dose proportional, with 0.04 nmoles having a significant but relatively minor effect. The duration of effect was approximately 24 to 48 hours, depending on dose. A 20 nmoles injection administered once i.p. did not significantly reduce food intake (data not shown).

Compounds (2) was also tested ICV at doses of approximately 40 nmoles in DMSO. As shown in FIG. 1B, Compound (2) altered subsequent 24 hour food intake. Finally, ouabain was also injected at approximately 8 nmoles in 4 μl DMSO. Ouabain resulted in motor hyperactivity, a previously reported phenomenon (See, Li, R., et al., (1997) Mol Chem Neuropathol, 31, 65-72), but had no consistent effect on food intake (results not shown).

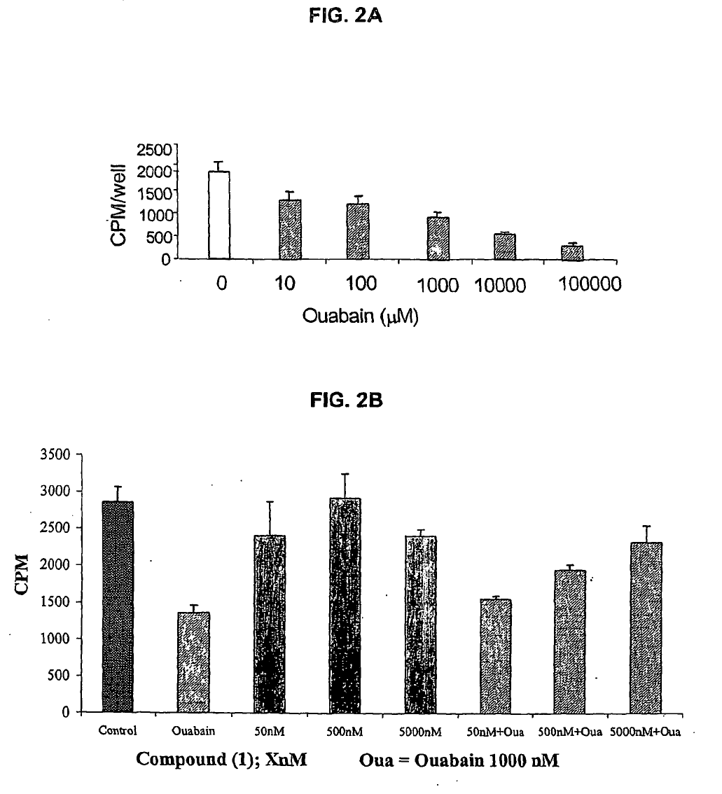

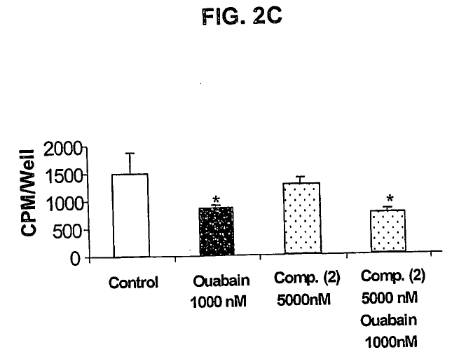

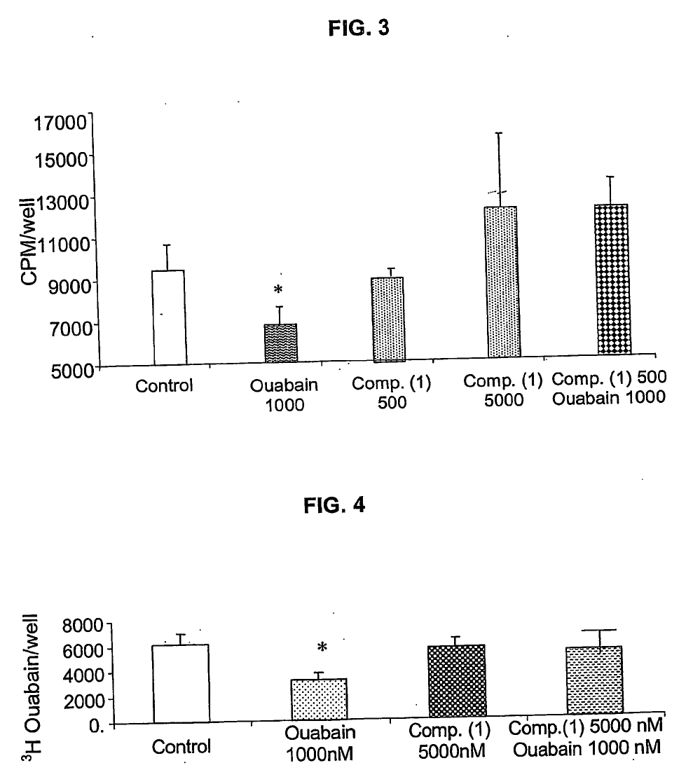

Effects of Compound (1) on ⁸⁶Rb Uptake in Hypothalamic Cultures and Explants in vitro (FIG. 2 and FIG. 3):

The test compounds have a 4 ring core and 14-0H substitution that suggest they are biochemically and phylogenetically related to other plant cardenolides; without the digitalis-specific D-ring lactone (see, e.g., Harborne, J. B. (2001) Nat Prod Rep, 18, 361-379; and LaBella, F. S., et al., (1998) Clin Exp Hypertens, 20, 601-609). For this reason, it was believed that the test compounds may interact with a cardiac glycoside binding site, e.g. on the Na/K ATPase complex, which in turn might result in anorexia (see, e.g., Lingrel, et al., (1994) Kidney Int Suppl, 44, S32-39; and Sweadrier, K. J., et al., (1980) N Engl J Med, 302, 777-783). Initial competitive binding studies in membrane concentrates from brain, kidney and gut, failed to demonstrate inhibition of ouabain binding or Na/K ATPase activity. However, in whole cell hypothalamic culture, Compound (1) inhibited the effect of ouabain on 86-rubidium uptake. Ouabain dose-dependently inhibits 86-Rb uptake into such cultures (FIG. 2A), an effect consistent with its inhibitory effect on Na/K exchange mediated by Na/K ATPase.

As shown in FIG. 2B, over a wide concentration range, Compound (1) alone had no effect on 86-Rb uptake. However, in a representative experiment in FIG. 2C, the compound significantly reduced the inhibition of 86-Rb uptake that occurs with ouabain alone. This activity was observed at concentrations between 5 and 500 nM. However, Compounds (2) (FIG. 2C) had no effect.

A similar effect of Compound (1) on ⁸⁶Rb uptake was also observed in incubated hypothalamic slice punches (FIG. 3). As expected, ouabain inhibited ⁸⁶Rb; whereas, Compound (1) alone at concentrations up to 5000 nM had no effect. However, as in the cultures, the compound almost completely blocked the inhibitory effect of ouabain.

Absence of Evidence of Binding to the Ouabain Receptor (FIG. 4):

Despite previous evidence cited above that the Compound (1) does not bind to or inhibit activity of membrane extracts enriched with Na/K ATPase, a whole cell assay system was used to confirm that Compound (1) did not bind to ouabain receptors. As shown in FIG. 4, tritiated (3H) ouabain binds to (extracted) cells in hypothalamic culture and is specifically inhibited by higher concentrations of unlabeled ouabain. This binding was unaffected and in some experiments appeared to actually increase during exposure to Compound (1). Compound (2) was not tested.

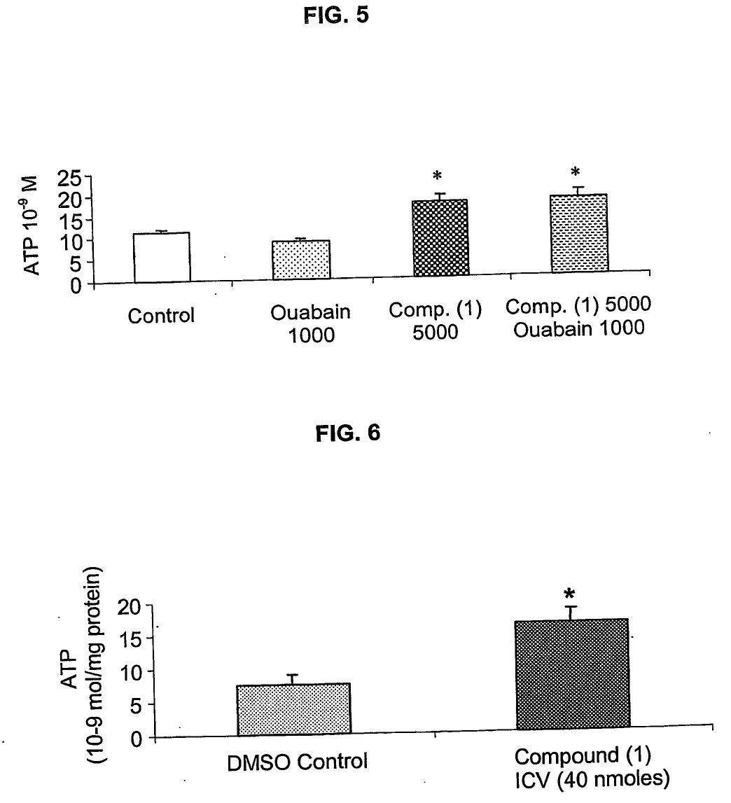

Effect of Compound (1) on ATP Content in Hypothalamic Culture or Slice Punch Explants (FIG. 5):

Although there are several possible explanations for the inhibition by Compound (1) of the inhibitory effect of ouabain on putative Na/K ATPase activity, it was presumed that this effect was not related to interference with ouabain binding nor to a direct toxic effect of the compound, for which there has been no in vivo or in vitro evidence. The Na/K ATPase exchanger or ‘pump’ is directly fueled by ATP and the activity of this exchanger is known to be modified by availability of ATP substrate (see, Dahl, J. L., et. al., (1974) Annu Rev Biochem, 43, 327-356). Accordingly, the ATP content in hypothalamic culture following incubation with the steroidal glycoside was measured. Ouabain alone had no effect or slightly reduced ATP content. 500 nM of Compound (1), either alone or in combination with ouabain, significantly increased ATP content by up to 2-fold following 30 minute incubations in vitro.

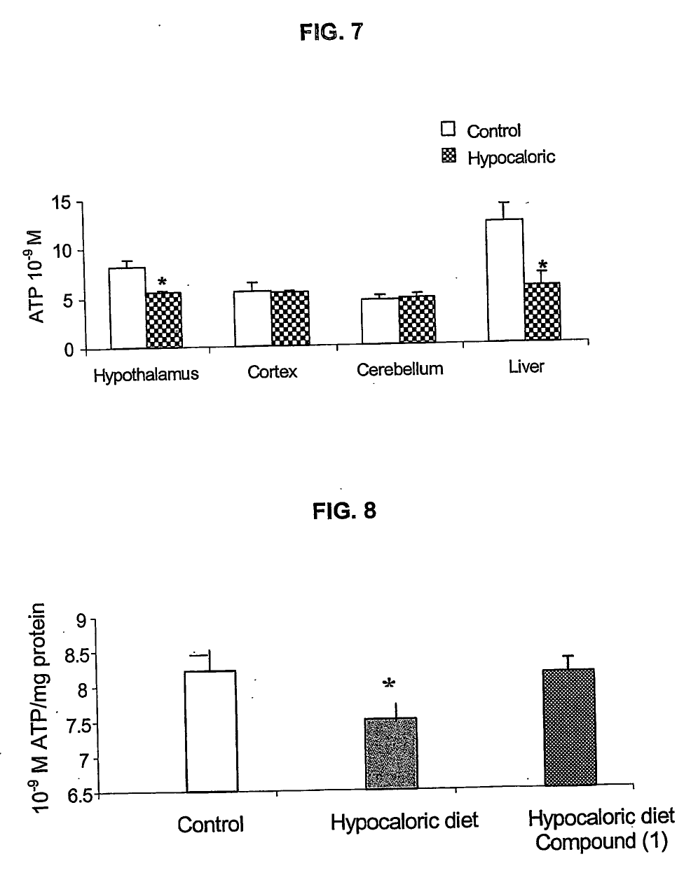

Effect of Compound (1) on Hypothalamic ATP Content in Rats Maintained on a Normal or on a Hypocaloric Diet (FIG. 6, FIG. 7 and FIG. 8):

To study the possible significance of altered hypothalamic ATP content in Compound (1)-treated rats, experiments were first performed to assess the effect of Compound (1) on hypothalamic ATP content and subsequently on animals maintained on a hypocaloric diet. Rats were maintained either on ad libitum or on a 4 day diet restricted to 5 grams per day plus ad libitum water. Animals were then sacrificed and regions of the brain and liver were sampled for ATP content.

In rats maintained on a normal diet, Compound (1) ICV injections increased hypothalamic ATP content (FIG. 6, P<0.05). In rats maintained for 4 days on a hypocaloric diet, ATP content in the basal hypothalamus (sampled from a larger basal hypothalamic section) was reduced by about 40% (P<0.001) (FIG. 7) Liver ATP content was also significantly reduced by about 60 percent; whereas, ATP content in the cortex and cerebellum were unaltered versus ad libitum fed rats.

In subsequent experiments, the 4 days of hypocaloric or eucaloric control diets were followed by ICV injection of 40 nmoles of Compound (1) or DMSO vehicle. Animals were maintained on their previous diets during the postoperative 24 hours. ATP content in hypothalamic slice punches was again reduced in hypocaloric/DMSO treated rats but was similar to ad libitum fed rats in those hypocaloric rats treated with ICV Compound (1) (FIG. 8).

### Pharmaceutical Etc. Formulations and Uses Thereof

Agents that modulate the production or content of ATP (referred to herein as an “ATP modulator”) in the hypothalamus are useful for treating metabolic diseases, disorders and conditions, for example appetitive diseases, disorders and/or conditions (e.g., obesity) and diabetes, and for causing weight loss without substantial adverse health effects.

Therefore, another embodiment of the present invention is a substance adapted for administration to an animal (“medicament”) and intended to treat metabolic diseases, disorders and conditions in the animal or to cause weight loss in the animal without substantial adverse health effects, the substance comprising a therapeutically effective amount of an agent known through (e.g. public) scientific research to modulate (e.g. increase) the production or content of ATP in the hypothalamus of the animal, optionally in association with a pharmaceutically acceptable or dietetic excipient, diluent or carrier, or a foodstuff or beverage.

The agent may, for example, be provided as fresh or preserved (e.g. dried) plant material containing the agent, or as a plant extract consisting of or containing the agent, as synthetic chemical compound or mixture of compounds, or any combination thereof. The plant extract may preferably be isolated and/or purified prior to use, e.g. by conventional isolation/purification methods.

The substance may be present with packaging and/or other written material describing the use and the activity of the agent in modulating the production or content of ATP in the hypothalamus.

A typical formulation is prepared by mixing the ATP modulator and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the ATP modulator is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wefting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., an ATP modulator or pharmaceutical composition thereof or aid in the manufacturing of the pharmaceutical product (i.e., medicament).

The formulations may be prepared using conventional dissolution and mixing procedures. For example, the bulk drug substance (i.e., ATP modulator or stabilized form of the ATP modulator (e.g., complex with a cyclodextrin derivative or other known complexation agent)) is dissolved in a suitable solvent in the presence of one or more of the excipients described above. The ATP modulator is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The present invention further provides a method of treating diseases, conditions and/or disorders modulated by ATP modulators in an animal that includes administering to an animal in need of such treatment a therapeutically effective amount of an ATP modulator or a pharmaceutical composition comprising an effective amount of an ATP modulator and a pharmaceutically acceptable excipient, diluent, or carrier. The method is particularly useful for treating diseases, conditions and/or disorders such as obesity and diabetes. Accordingly, ATP modulators (including the compositions and processes used therein) may be used in the manufacture of a medicament for the therapeutic applications described herein.

The ATP modulators can be administered to a patient at dosage levels in the range of from about 0.7 mg to about 7,000 mg per day. For an adult human having a body weight of about 70 kg, a dosage in the range of from about 0.01 mg to about 100 mg per kilogram body weight is typically sufficient. However, some variability in the general dosage range may be required depending upon the age and weight of the subject being treated, the intended route of administration, the particular ATP modulator or additional agent being administered and the like. The determination of dosage ranges and optimal dosages for a particular patient is well within the ability of one of ordinary skill in the art having the benefit of the instant disclosure. It is also noted that the compounds of the present invention can be used in sustained release, controlled release, and delayed release formulations, which forms are also well known to one of ordinary skill in the art.

The ATP modulators may also be used in conjunction with other pharmaceutical agents for the treatment of the diseases, conditions and/or disorders described herein. Therefore, methods of treatment that include administering ATP modulators in combination with other pharmaceutical agents are also provided. Suitable pharmaceutical agents that may be used in combination with the ATP modulators include anti-obesity agents such as apolipoprotein-B secretion/microsomal triglyceride transfer protein (apo-B/MTP) inhibitors, MCR4 agonists, cholecystokinin-A (CCK-A) agonists, monoamine reuptake inhibitors (such as sibutramine), sympathomimetic agents, β₃ adrenergic receptor agonists, dopamine agonists (such as bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y antagonists, thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin receptor antagonists, histamine 3 receptor antagonists or inverse agonists, neuromedin U receptor agonists and the like. Other anti-obesity agents, including the preferred agents set forth hereinbelow, are well known, or will be readily apparent in light of the instant disclosure, to one of ordinary skill in the art.

Especially preferred are anti-obesity agents selected from the group consisting of orlistat, sibutramine, bromocriptine, ephedrine, leptin, and pseudoephedrine. Preferably, compounds of the present invention and combination therapies are administered in conjunction with exercise and a sensible diet.

Representative anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Pat. Nos. 3,752,814 and 3,752,888; and orlistat can be prepared as described in U.S. Pat. Nos. 5,274,143; 5,420,305; 5,540,917; and 5,643,874. All of the above recited U.S. patents are incorporated herein by reference.

Other pharmaceutical agents that may be useful include antihypertensive agents; antidepressants; insulin and insulin analogs (e.g., LysPro insulin); GLP-1 (7-37) (insulinotropin) and GLP-1 (7-36)-NH₂; sulfonylureas and analogs thereof chlorpropamide, glibenclamide, tolbutamide, tolazamide, acetohexamide, Glypizide®, glimepiride, repaglinide, meglitinide; biguanides: metformin, phenformin, buformin; α2-antagonists and imidazolines: midaglizole, isaglidole, deriglidole, idazoxan, efaroxan, fluparoxan; other insulin secretagogues: linogliride, A-4166; glitazones: ciglitazone, Actos® (pioglitazone), englitazone, troglitazone, darglitazone, Avandia® (BRL49653); fatty acid oxidation inhibitors: clomoxir, etomoxir; α-glucosidase inhibitors: acarbose, miglitol, emiglitate, voglibose, MDL-25,637, camiglibose, MDL-73,945; β-agonists: BRL 35135, BRL 37344, RO 16-8714, ICI D7114, CL 316,243; phosphodiesterase inhibitors: L-386,398; lipid-lowering agents: benfluorex: fenfluramine; vanadate and vanadium complexes (e.g., Naglivan®) and peroxovanadium complexes; amylin antagonists; glucagon antagonists; gluconeogenesis inhibitors; somatostatin analogs; antilipolytic agents: nicotinic acid, acipimox, WAG 994, pramlintide (Symlin™), AC 2993, nateglinide, aldose reductase inhibitors (e.g., zopolrestat), glycogen phosphorylase inhibitors, sorbitol dehydrogenase inhibitors, sodium-hydrogen exchanger type 1 (NHE-1) inhibitors and/or cholesterol biosynthesis inhibitors or cholesterol absorption inhibitors, especially a HMG-CoA reductase inhibitor, or a HMG-CoA synthase inhibitor, or a HMG-CoA reductase or synthase gene expression inhibitor, a CETP inhibitor, a bile acid sequesterant, a fibrate, an ACAT inhibitor, a squalene synthetase inhibitor, an anti-oxidant or niacin. The ATP modulators may also be administered in combination with a naturally occurring compound that acts to lower plasma cholesterol levels. Such naturally occurring compounds are commonly called nutraceuticals and include, for example, garlic extract and niacin.

The dosage of the additional pharmaceutical agent is generally dependent upon a number of factors including the health of the subject being treated, the extent of treatment desired, the nature and kind of concurrent therapy, if any, and the frequency of treatment and the nature of the effect desired. In general, the dosage range of the additional pharmaceutical agent is in the range of from about 0.001 mg to about 100 mg per kilogram body weight of the individual per day, preferably from about 0.1 mg to about 10 mg per kilogram body weight of the individual per day. However, some variability in the general dosage range may also be required depending upon the age and weight of the subject being treated, the intended route of administration, the particular anti-obesity agent being administered and the like. The determination of dosage ranges and optimal dosages for a particular patient is also well within the ability of one of ordinary skill in the art having the benefit of the instant disclosure.

According to the present invention, an ATP modulator or a combination of an ATP modulator and optionally at least one additional pharmaceutical agent is administered to a subject in need of such treatment, preferably in the form of a pharmaceutical composition. In the combination aspect of the invention, the ATP modulator and at least one other pharmaceutical agent (e.g., anti-obesity agent) may be administered either separately or in the pharmaceutical composition comprising both. It is generally preferred that such administration be oral. However, if the subject being treated is unable to swallow, or oral administration is otherwise impaired or undesirable, parenteral or transdermal administration may be appropriate.

According to the present invention, when a combination of an ATP modulator and at least one other pharmaceutical agent are administered together, such administration can be sequential in time or simultaneous with the simultaneous method being generally preferred. For sequential administration, an ATP modulator and the additional pharmaceutical agent can be administered in any order. It is generally preferred that such administration be oral. It is especially preferred that such administration be oral and simultaneous. When an ATP modulator and the additional pharmaceutical agent are administered sequentially, the administration of each can be by the same or by different methods.

According to the present invention, an ATP modulator or a combination of an ATP modulator and at least one additional pharmaceutical agent (referred to herein as a “combination”) is preferably administered in the form of a pharmaceutical composition. Accordingly, an ATP modulator or a combination can be administered to a patient separately or together in any conventional oral, rectal, transdermal, parenteral, (for example, intravenous, intramuscular, or subcutaneous) intracisternal, intravaginal, intraperitoneal, intravesical, local (for example, powder, ointment or drop), or buccal, or nasal, dosage form.

Compositions suitable for parenteral injection generally include pharmaceutically acceptable sterile aqueous or nonaqueous solutions, dispersions, suspensions, or emulsions, and sterile powders for reconstitution into sterile injectable solutions or dispersions. Examples of suitable aqueous and nonaqueous carriers or diluents (including solvents and vehicles) include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain excipients such as preserving, wetting, emulsifying, and dispersing agents. Prevention of microorganism contamination of the compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and gelatin.

Solid dosage forms for oral administration include capsules, tablets, powders, and granules. In such solid dosage forms, an ATP modulator or a combination is admixed with at least one inert customary pharmaceutical excipient (or carrier) such as sodium citrate or dicalcium phosphate or (a) fillers or extenders (e.g., starches, lactose, sucrose, mannitol, silicic acid and the like); (b) binders (e.g., carboxymethylcellulose, alginates, gelatin, polyvinylpyrrolidone, sucrose, acacia and the like); (c) humectants (e.g., glycerol and the like); (d) disintegrating agents (e.g., agar-agar, calcium carbonate, potato or tapioca starch, alginic acid, certain complex silicates, sodium carbonate and the like); (e) solution retarders (e.g., paraffin and the like); (f) absorption accelerators (e.g., quaternary ammonium compounds and the like); (g) wetting agents (e.g., cetyl alcohol, glycerol monostearate and the like); (h) adsorbents (e.g., kaolin, bentonite and the like); and/or (i) lubricants (e.g., talc, calcium stearate, magnesium stearate, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings and shells, such as enteric coatings and others well known in the art. They may also contain opacifying agents, and can also be of such composition that they release the ATP modulator and/or the additional pharmaceutical agent in a delayed manner. Examples of embedding compositions that can be used are polymeric substances and waxes. The drug can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the ATP modulator or the combination, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), glycerol, tetrahydrofurfuryl alcohol, polyethylene glycols and fatty acid esters of sorbitan, or mixtures of these substances, and the like.

Besides such inert diluents, the composition can also include excipients, such as wetting agents, emulsifying and suspending agents, sweetening, flavoring, and perfuming agents.

Suspensions, in addition to the ATP modulator or the combination, may further comprise suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by mixing an ATP modulator or a combination with suitable non-irritating excipients or carriers, such as cocoa butter, polyethylene glycol or a suppository wax which are solid at ordinary room temperature but liquid at body temperature and therefore melt in the rectum or vaginal cavity thereby releasing the active component(s).

Dosage forms for topical administration of the ATP modulators and combinations with anti-obesity agents may comprise ointments, powders, sprays and inhalants. The drugs are admixed under sterile condition with a pharmaceutically acceptable carrier, and any preservatives, buffers, or propellants that may be required. Ophthalmic formulations, eye ointments, powders, and solutions are also intended to be included within the scope of the present invention.

The following paragraphs describe exemplary formulations, dosages, etc. useful for non-human animals. The administration of the ATP modulators and combinations can be effected orally or non-orally (e.g., by injection).

The amount of ATP modulator or combination is administered such that an effective dose is received. Generally, a daily dose that is administered orally to an animal is between about 0.01 and about 1,000 mg/kg of body weight, preferably between about 0.01 and about 300 mg/kg of body weight.

Conveniently, the ATP modulator (or combination) can be carried in the drinking water so that a therapeutic dosage of the ATP modulator is ingested with the daily water supply. The ATP modulator can be directly metered into drinking water, preferably in the form of a liquid, water-soluble concentrate (such as an aqueous solution of a water-soluble salt).

Conveniently, an ATP modulator (or combination) can also be added directly to the feed, as such, or in the form of an animal feed supplement, also referred to as a premix or concentrate. A premix or concentrate of the ATP modulator in a carrier is more commonly employed for the inclusion of the agent in the feed. Suitable carriers are liquid or solid, as desired, such as water, various meals such as alfalfa meal, soybean meal, cottonseed oil meal, linseed oil meal, corncob meal and corn meal, molasses, urea, bone meal, and mineral mixes such as are commonly employed in poultry feeds. A particularly effective carrier is the respective animal feed itself; that is, a small portion of such feed. The carrier facilitates uniform distribution of the ATP modulator in the finished feed with which the premix is blended. Preferably, the ATP modulator is thoroughly blended into the premix and, subsequently, the feed. In this respect, the ATP modulator may be dispersed or dissolved in a suitable oily vehicle such as soybean oil, corn oil, cottonseed oil, and the like, or in a volatile organic solvent and then blended with the carrier. It will be appreciated that the proportions of ATP modulator in the concentrate are capable of wide variation since the amount of the ATP modulator in the finished feed may be adjusted by blending the appropriate proportion of premix with the feed to obtain a desired level of compound.

High potency concentrates may be blended by the feed manufacturer with proteinaceous carrier such as soybean oil meal and other meals, as described above, to produce concentrated supplements, which are suitable for direct feeding to animals. In such instances, the animals are permitted to consume the usual diet. Alternatively, such concentrated supplements may be added directly to the feed to produce a nutritionally balanced, finished feed containing a therapeutically effective level of an ATP modulator. The mixtures are thoroughly blended by standard procedures, such as in a twin shell blender, to ensure homogeneity.

If the supplement is used as a top dressing for the feed, it likewise helps to ensure uniformity of distribution of the ATP modulator across the top of the dressed feed.

Drinking water and feed effective for increasing lean meat deposition and for improving lean meat to fat ratio are generally prepared by mixing an ATP modulator with a sufficient amount of animal feed to provide from about 10⁻³ to about 500 ppm of the ATP modulator in the feed or water.

The preferred medicated swine, cattle, sheep and goat feed generally contain from about 1 to about 400 grams of an ATP modulator (or combination) per ton of feed, the optimum amount for these animals usually being about 50 to about 300 grams per ton of feed.

The preferred poultry and domestic pet feeds usually contain about 1 to about 400 grams and preferably about 10 to about 400 grams of an ATP modulator (or combination) per ton of feed.

For parenteral administration in animals, the ATP modulators (or combination) may be prepared in the form of a paste or a pellet and administered as an implant, usually under the skin of the head or ear of the animal in which increase in lean meat deposition and improvement in lean meat to fat ratio is sought.

In general, parenteral administration involves injection of a sufficient amount of an ATP modulator (or combination) to provide the animal with about 0.01 to about 20 mg/kg/day of body weight of the drug. The preferred dosage for poultry, swine, cattle, sheep, goats and domestic pets is in the range of from about 0.05 to about 10 mg/kg/day of body weight of drug.

Paste formulations can be prepared by dispersing the drug in a pharmaceutically acceptable oil such as peanut oil, sesame oil, corn oil or the like.

Pellets containing an effective amount of an ATP modulator, pharmaceutical composition, or combination can be prepared by admixing an ATP modulator or combination with a diluent such as carbowax, carnuba wax, and the like, and a lubricant, such as magnesium or calcium stearate, can be added to improve the pelleting process.

It is, of course, recognized that more than one pellet may be administered to an animal to achieve the desired dose level which will provide the increase in lean meat deposition and improvement in lean meat to fat ratio desired. Moreover, implants may also be made periodically during the animal treatment period in order to maintain the proper drug level in the animal's body.

The present invention has several advantageous veterinary features. For the pet owner or veterinarian who wishes to increase leanness and/or trim unwanted fat from pet animals, the instant invention provides the means by which this may be accomplished. For poultry and swine breeders, utilization of the method of the present invention yields leaner animals that command higher sale prices from the meat industry.

1. A method for the identification of agents for use in the treatment of metabolic diseases, disorders or conditions in an animal in need of such treatment, comprising the step of identifying an agent that modulates ATP production or content in the hypothalamus of the animal.

2. A method according to claim 1, wherein the metabolic disease, disorder or condition is an appetitive disease, disorder or condition.

3. A method according to claim 2, wherein the metabolic disease, disorder or condition is obesity.

4. A method according to claim 2, wherein the metabolic disease, disorder or condition is diabetes.

5. A method comprising the step of identifying an agent that modulates ATP production or content in the hypothalamus of the animal, wherein said method identified agents that:
a. regulate food-intake or calorific-intake in the animal, or
b. decrease food-intake or calorific-intake in an animal, or
c. cause body mass reduction in an animal without substantial adverse health effects, or
d. aid in the treatment of diabetes in an animal in need of such treatment.

6. (canceled)

7. (canceled)

8. A method according to claim 5, wherein the agent increases ATP production or content in the hypothalamus of the animal.

9. A method for the identification of agents for use in the treatment of diabetes in an animal in need of such treatment, comprising the step of identifying an agent that modulates ATP production or content in the hypothalamus of the animal.

10. (canceled)

11. A method according to claim 1, further including the step, after identification of an agent that modulates ATP production or content in the hypothalamus of an animal, of identifying a source of the agent.

12. A method according to claim 11, wherein the source is a plant or a plant extract.

13. A method according to claim 11, wherein the source is a synthetically produced agent.

14. A method according to claim 1, wherein the agent is a steroidal glycoside.

15. A method according to claim 14, wherein the steroidal glycoside comprises a mono- or poly-saccharide moiety at the 3-O position of the steroid ring system.

16. A method according to claim 15, wherein the mono- or poly-saccharide moiety is a trisaccharide comprising 6-deoxy and/or 2,6-dideoxy hexoses.

17. A method according to claim 16, wherein the terminal saccharide of the mono- or poly-saccharide moiety is thevetosyl.

18. A method according to claim 14, wherein the steroidal glycoside comprises an ester moiety at the 12 position of the steroid ring system.

19. A method according to claim 18, wherein the ester is benzoyl, tigloyl or anthraninoyl.

20. A method according to claim 1, further including the step, after identifying the source of the agent, of providing the source of the agent as a substance adapted for administration to the animal.

21. A method according to claim 20, wherein the substance is fresh, dried or otherwise preserved plant material.

22. A method according to claim 20, wherein the substance is an isolated and/or purified plant extract.

23. A method according to claim 20, wherein the substance is a pharmaceutical composition.

24. A method according to claim 20, wherein the substance is a foodstuff or food additive.

25. A method according to claim 20, wherein the substance is a beverage or beverage additive.

26. A method for treating a metabolic disease, disorder or condition in an animal in need of such treatment, comprising administering a therapeutically effective amount of an agent known through scientific research to modulate ATP production or content in the hypothalamus of the animal, and for the purpose of knowingly modulating ATP production or content in the hypothalamus of the animal.

27. A pharmaceutical composition comprising a therapeutically effective amount of an agent known through scientific research to modulate ATP production or content in the hypothalamus of an animal, wherein said composition is a-medicament for treating a metabolic disease, disorder or condition in the animal in need of such treatment by knowingly modulating ATP production or content in the hypothalamus of the animal.

28. (canceled)

29. (canceled)

30. (canceled)

31. (canceled)

32. A method for causing body mass reduction in an animal without substantial adverse health effects, comprising administering a therapeutically effective amount of an agent known through scientific research to modulate ATP production or content in the hypothalamus of the animal, and for the purpose of knowingly modulating ATP production or content in the hypothalamus of the animal.

33. A pharmaceutical composition comprising a therapeutically effective amount of an agent known through scientific research to modulate ATP production or content in the hypothalamus of an animal, wherein said composition is a medicament for causing body mass reduction in the animal without substantial adverse health effects by knowingly modulating ATP production or content in the hypothalamus of the animal.

34. A substance adapted for administration to an animal and intended to treat metabolic diseases, disorders and conditions in the animal or to cause weight loss in the animal without substantial adverse health effects, the substance comprising a therapeutically effective amount of an agent known through scientific research to modulate the production or content of ATP in the hypothalamus of the animal, optionally in association with a pharmaceutically acceptable or dietetic excipient, diluent or carrier, or a foodstuff or beverage.

35. A substance according to claim 34, wherein the agent is provided as fresh or preserved plant material containing the agent.

36. A substance according to claim 34, wherein the agent is provided as a plant extract consisting of or containing the agent.

37. A substance according to claim 34, wherein the agent is provided as a synthetic chemical compound or mixture of compounds.

38. A substance according to claim 34, when present with packaging and/or other written material describing the use and the activity of the agent in modulating the production or content of ATP in the hypothalamus.

39. A method according to claim 26, wherein the agent is a steroidal glycoside.

40. A pharmaceutical composition according to claim 27, wherein the agent is a steroidal glycoside.

41. A substance according to claim 34, wherein the agent is a steroidal glycoside.

42. A method according to claim 26, wherein the disease, disorder or condition to be treated, or the physiological effect to be caused, is an appetitive disease, disorder or condition.

43. A pharmaceutical composition according to claim 27, wherein the disease, disorder or condition to be treated, or the physiological effect to be caused, is an appetitive diseases disorder or condition.

44. A substance according to claim 34, wherein the disease, disorder or condition to be treated, or the physiological effect to be caused, is an appetitive disease, disorder or condition.

45. A method for the identification of agents for use in the treatment of appetitive diseases, disorders, or conditions in an animal comprising the step of identifying an agent that modulates ATP production in the hypothalamus of said animal.

46. A method for the identification of agents that regulate food-intake in an animal comprising the step of identifying an agent that modulates ATP production in the hypothalamus of said animal.

47. The method of claim 46, wherein the regulation of food-intake is a decrease in food-intake.

48. A method for the identification of agents for use in the treatment of diabetes in an animal comprising the step of identifying an agent that increases ATP production or content in the hypothalamus of said animal.

49. A method for treating an appetitive disease, disorder, or condition comprising the step of administering to an animal in need of such treatment a therapeutically effective amount of an agent that modulates ATP production in the hypothalamus of an animal, provided that said agent is not an extract from a plant of the genus Trichocaulon or Hoodia.

50. (canceled)

51. A method for treating diabetes comprising the step of administering to an animal in need of such treatment a therapeutically effective amount of an agent that increases ATP production or content in the hypothalamus of an animal, provided that said agent is not an extract from a plant of the genus Trichocaulon or Hoodia.

52. A pharmaceutical composition comprising therapeutically effective amount of an agent that modulates ATP production in the hypothalamus of an animal, wherein the composition is a medicament for treating an appetitive disease, disorder, or condition in an animal in need of such treatment, provided that said agent is not an extract from a plant of the genus Trichocaulon or Hoodia.

53. (canceled)

54. A pharmaceutical composition comprising therapeutically effective amount of an agent that increases ATP production or content in the hypothalamus of an animal, wherein the composition is a medicament for treating diabetes in an animal in need of such treatment, provided that said agent is not an extract from a plant of the genus Trichocaulon or Hoodia.

55. The method of claim 26, wherein the metabolic disease, disorder, or condition is obesity.

56. The pharmaceutical composition of claim 55, wherein the metabolic disease, disorder or condition is obesity.

57. The method of claim 26, wherein the metabolic disease, disorder or condition is diabetes.

58. The pharmaceutical composition of claim 55, wherein the metabolic disease, condition or disorder is diabetes.

59. The method of claim 49, wherein the appetitive disease, disorder, or condition is obesity.

60. The pharmaceutical composition of claim 52, wherein the appetitive disease, disorder or condition is obesity.
